SEQUENCE LISTING (5 sequences)

SEQ ID NO: 1 - DNA, 3750 bp, Homo sapiens
  misc_feature (39)..(39): R represents a polymorphorism of a change of base from G to A
ggaaggatacttatgaagttcatttaaagagaattactrtttccaaagttcttgatttatgaagcaggcattcaatttcaagatgtaaaataatagtgtaagttggttggggtcgggggagaccatcatcatatgattatacagattttcccaatttttttcctcaatgaaatagcttattttaaaaatataaaattagaatccctataaagaacactaccaattttctttcctgaaagaaacaataaaatactttgtaaatagaagcaacttcatgaagtaacagatgttttatgtaaagacataaaactgaacctgaatataacgagacgtgtgtgcgagtagtaaaagttgaaagttggacacactcattgagacatatctatttgattccaatgtttttctaaaaggtagagtaatcctagccagaggtttcactggctcagtgcatcacccagtagtgtctcagaagccaggaagggctttccattagataatgaattatgaaatgtctcacactggaaaaaccagtcatccgctgatgtcatgctgattccaaccaatcccaaacaaagccccagccctcctctgtttcagtggtaccaatgtgtggtgtacaaataagtagtacagtataaaacttcacagtgccaataccatgaagaggagctcagacagctcttaccacatgatacaagagccggctggtggaagagtggggaccagaaaggtaatgctttttaactcttacttctgagctctttacacattcaaagataggaaagctaggaggaattttacaactaattggcatttccaatgtgcattgtgatgtgtacctttttatattattcaggcaggttaatacagcttttaatagtcctagagcatgcaaatagattatatgtttatacaagccactcagcacatatatacaagtacatatgccaaagagaaagctatttttaagagttacattcgcaaacagtaaattcagggaacacacacatactcagatgcagagagaatccaaatattgataagttgcacttatctaaatgctgctattaggactcctgagttgtttagagccattaaacttttggttgtatttcagactttcttgtaaaacttaattgaactgcaaaacattttgggtactgtatatgtgactccaaataggtggatgatgttaagtattatagcacaaagattttttataaaaccattgtaacacaaatgtcccctgcctcccccattctctttcaccatccccgtaaaaaatatgaggctttttaggcaatgttgacaaagttttaacaataatggtggagtaattgatgtttctggagctgaaaccccaaaggtgttaggttacttgtaacagaaaaagtcttacaaatagttgttttggaaaaggggacagtatatataattcagaaagcattgttaacctgtgcaaactgtaattataacttagtgtcacaattttcttgccctcttccccttgcactcagatcttatcttgtagtaatgatatttattaactctttccacctaaactgcattgcttgactgtaatgctatgaacacactaggtgtcagatataagctgagtgtatcttcagaaaccaagagggcttatgtgtgggaaagaaaccgagagggaaggaacgctttaacagatggaccccttaaagattcttctgcaagataaaagcaataagacagaaaatgaaaaagaggggagggggaagaattttttttaagccttagaaaggcattgttaaaaaattcacatttttctttttctgtgcacactaaaatccatgatgatttcatctgcactgttcctttgagggaaaaagaagcagtcaaggaggctgtctatgaatgcactggtcgggacaggcttggggcaagctgaaaaaactaccacatgacagagaaaaataatttgccaatatattttagagagtcttttcccataggaccagttattcaagtcatacgagtgcactctttttataaaaggatgtgggaaaggccaagagaattttgcattttatctgtgaagtccggcgagtggtggtaggctgtaatgtgtgagagtgagtgggtccccggcagaagggggcagctgaacgcgacggggagaaagcgcttctggaacttgggcttgtgacaggctgcctgccctctggtccttcagtgccctgctgcatttcacagttgggaagagtggagtgtattatatgaccccaaacaaaagttccattgcgctctcctcagattgcctgccagtgttgatgacctgaacatttaaatatgaatgaattgggggaaaggaccatctcccctggatcccatcaggccagaacaatcctctgttacccctgagtccctcttccctgacctccactacctgtccactggacctccctgcaccctctgccccaccacgtggccaggtgggcgttctaccacctagggctgtggcttggctgggtggaggggcggtgggaacactttttcaatcaattcatccctattgattgagacactgtgtttgtttggggtttcttttctcccctccaaaaaaggaagaaggtgaaaccaggagactgggcagagaaagaaaaaaaaatagtgaacaaaattaggataatttattttagaagagaaagtagaacccccgaaaatggtgatatttgaagagaggtgtctgtgaggaagctaagagcagaaggagagcagcctgtcagaaaacgggctgtccctccctccctaatcacagccctactcacagcaaactccctccctctccattcactcacttacttagggccaatcctttctctctctctctctctctctctctctctctctctcttcctttccctctctccctctccccctcccttccctccctctctccctctctctcccccttctttccctctctcttctctccccctctctcctctctctctgtctctgtctccctcccatcctctctctctgtctctgtctgtctccccgccaccctgtctctccctccctccctgtctccctccctccctccctccttccttctctcttactcggagacagtcagaactctcctccctgacagccacaaacctacagcactgactgcattcagagaggaacctgcaaacaaaacttcacagaaaactttttgttcttgttccagagaatttgctgaagaggagaaggaaaaaaaaaacaccaaaaaaaaaaataaaaaaatccacacacacaaaaaaacctgcgcgtgaggggggaggaaaagcagggccttttaaaaaggcaatcacaacaacttttgctgccaggatgcccttgctttggctgagaggatttctgttggcaagttgctggattatagtgaggagttcccccaccccaggatccgaggggcacagcgcggcccccgactgtccgtcctgtgcgctggccgccctcccaaaggatgtacccaactctcagccagagatggtggaggccgtcaagaagcacattttaaacatgctgcacttgaagaagagacccgatgtcacccagccggtacccaaggcggcgcttctgaacgcgatcagaaagcttcatgtgggcaaagtcggggagaacgggtatgtggagatagaggatgacattggaaggagggcagaaatgaatgaacttatggagcagacctcggagatcatcacgtttgccgagtcaggt

SEQ ID NO: 2 - DNA, 1140 bp, Homo sapiens
  misc_feature (367)..(367): R represents a polymorphorism of a change of base from G to A
gtcatgagaactgggcttatgcttcaactgtagtccttcaaacaacccttcaccgttcttgtcagaggaccccttcctcctgtgttatttaccagacttgccaacacccctcccccacaaaaaaagcctgaccagctttacccctatttcaaggagtgttcacttcttttcattaatctgctgttatatttgtaacaatttagaaattataggagttgaagttctggtaaaaaagaatgatgcatgagggtttttgttgttgttgttgttgtgtgtgtgtgtgtgtgtgtgtgtgtttggttagttagaatgagattctaaagacctgggaaggatacttatgaagttcatttaaagagaattactrtttccaaagttcttgatttatgaagcaggcattcaatttcaagatgtaaaataatagtgtaagttggttggggtcgggggagaccatcatcatatgattatacagattttcccaatttttttcctcaatgaaatagcttattttaaaaatataaaattagaatccctataaagaacactaccaattttctttcctgaaagaaacaataaaatactttgtaaatagaagcaacttcatgaagtaacagatgttttatgtaaagacataaaactgaacctgaatataaygagacgtgtgtgcgagtagtaaaagttgaaagttggacacactcattgagacatatctatttgattccaatgtttttctaaaaggtagagtaatcctagccagaggtttcactggctcagtgcatcacccagtagtgtctcagaagccaggaagggctttccattagataatgaattatgaaatgtctcacactggaaaaaccagtcatccgctgatgtcatgctgattccaaccaatcccaaacaaagccccagccctcctctgtttcagtggtaccaatgtgtggtgtacaaataagtagtacagtataaaacttcacagtgccaataccatgaagaggagctcagrcagctcttaccacatgatacaagagccggctggtggaagagtggggaccagaaaggtaatgctttttaactcttacttctgagctctttacacattcaaagataggaaagctaggaggaattttacaactaattggc

SEQ ID NO: 3 - DNA, 19 bp, Artificial Sequence
  Synthetic sequence PSQ assay unlabelled PCR oligonucleotide
gaccccaaccaacttacac

SEQ ID NO: 4 - DNA, 22 bp, Artificial Sequence
  Synthetic sequence PSQ assay biotin PCR oligonucleotide
gagattctaaagacctgggaag

SEQ ID NO: 5 - DNA, 20 bp, Artificial Sequence
  Synthetic sequence PSQ assay sequencing oligonucleotide
cataaatcaagaactttgga

(19) United States
(12) Patent Application Publication
(10) Pub. No.: US 2006/0211774 A1
(43) Pub. Date: Sep. 21, 2006

(54) Method of diagnosins a genetic susceptibility for bone damage

(76) Inventors: Simon Thomas Bennett, Oxfordshire (GB); Mark Edwards, Ayleburg Bucha (GB)

Correspondence Address: HOFFMANN & BARON, LLP, 6900 JERICHO TURNPIKE, SYOSSET, NY, 11791, US

(21) Appl. No.: 10/559,294

(22) PCT Filed: Apr. 29, 2004
(86) PCT No.: PCT/GB04/01848

§ 371 Date: Nov. 30, 2005

(30) Foreign Application Priority Data
Apr. 29, 2003 (GB) 0309776.3

(51) Int. Cl. A61K 31/165

(52) U.S. Cl. 514/867; 514/622

(57) ABSTRACT

The present invention relates to the identification of a novel nucleotide polymorphism in the inhibin beta-A (INHBA) gene and the exploitation of this nucleotide polymorphism in the diagnosis of susceptibility to bone damage, particularly fracture. Also provided are transgenic non-human animals comprising the polynucleotides of the present invention and methods and kits for diagnosing and/or determining susceptibility to bone damage.

The present invention relates to the identification of a novel nucleotide polymorphism in the inhibin beta-A (INHBA) gene and the exploitation of this nucleotide polymorphism in the diagnosis of susceptibility to bone damage, particularly fracture. Also provided are transgenic non-human animals comprising the polynucleotides of the present invention and methods and kits for diagnosing and/or determining susceptibility to bone damage.

The inhibin beta A subunit joins the alpha subunit to form a pituitary FSH secretion inhibitor. Inhibin has been shown to regulate gonadal stromal cell proliferation negatively and to have tumour-suppressor activity. In addition, serum levels of inhibin have been shown to reflect the size of granulosa-cell tumors and can therefore be used as a marker for primary as well as recurrent disease. Because expression in gonadal and various extragonadal tissues may vary several fold in a tissue-specific fashion, it is proposed that inhibin may be both a growth/differentiation factor and a hormone. Furthermore, the beta A subunit forms a homodimer, activin A, and also joins with a beta B subunit to form a heterodimer, activin AB, both of which stimulate FSH secretion. Finally, it has been shown that the beta A subunit mRNA is identical to the erythroid differentiation factor subunit mRNA and that only one gene for this mRNA exists in the human genome.

Osteoporosis is a common disease characterized by reduced bone mineral density (BMD), deterioration of bone micro-architecture and increased risk of bone damage, such as fracture. It is a major public health problem which affects quality of life and increases costs to health care providers. In European populations, one in three women and one in twelve men over the age of fifty is at risk. The disease affects 25 million people in the USA, where the incidence of disease is 25% higher than it is in the UK, and a further 50 million people in Japan and Europe combined. It is estimated that by the middle of the next century the number of osteoporosis sufferers will double in the West, but may increase six-fold in Asia and South-America Fracture is the most serious endpoint of osteoporosis, particularly fracture of the hip which affects up to 1.7 million people worldwide each year. It is estimated that by the year 2050, the number of hip fractures worldwide will increase to over 6 million, as life expectancy and age of the population increase.

Treatment of osteoporosis is unsatisfactory. In particular, once bone damage has occurred as a result of osteoporosis, there is little a physician can do other than let the bone heal. In the elderly, this may be a slow and painful process. Diagnosis of those at risk of developing osteoporosis allows more effective preventative measures. Strategies for the prevention of this disease include development of bone density in early adulthood, and minimisation of bone loss in later life. Changes in lifestyle, nutrition and hormonal factors have been shown to affect bone loss.

Osteoporosis can be considered a complex genetic trait with variants of several genes underlying the genetic determination of the variability of the phenotype. Low bone mineral density is an important risk factor for fractures, the clinically most relevant feature of osteoporosis.

In view of the multigenic control of bone mineral density and therefore disease states such as osteoporosis, it is apparent that the identification of additional genetic factors which contribute to susceptibility to bone damage will provide important new insights into such disease, and may enable preventative measures to be applied, and new diagnostic and/or prognostic tests and treatments to be developed.

Testing for genetic susceptibility is an important enabling diagnostic tool. In many cases, the preferred test will be for more than one factor involved in a disease, e.g. osteoporosis. This provides a broader picture, with increased information, on the genetic susceptibility of a male or female individual. Genetic susceptibility is the risk that an individual either suffers from, or will suffer from, a disease.

The present invention provides, amongst other aspects a method of determining a male or female individual's susceptibility to bone damage.

In a first aspect, the present invention provides a method of determining susceptibility to bone damage, comprising determining the presence or absence of a polymorphism at position 39 of at least one allele of the INHBA gene. In a specific embodiment the polymorphism consists of the presence of nucleotide base A at position 39.

The method of the first aspect may be practised on any mammalian subject. Preferably, the mammalian subject will be a human, and most preferably an adult, preferably female, alternatively male.

The novel polymorphism in the INHBA gene has been shown to be responsible for increased susceptibility to bone damage and related conditions such as osteoporosis. In particular, the polymorphism of the present invention, either alone or in combination with other polymorphisms, is useful in identifying individuals, either male or female, susceptible to or resistant to bone damage and in the prevention and/or treatment of this condition.

The present invention is applicable to any disease in which bone damage is a factor, such as osteoporosis and osteoporotic fracture. Bone damage may be defined as any form of damage resulting from low bone mineral density, and includes any form of structural damage, such as fractures, bones or chips, and degradation or deterioration of the bone other than normal wear and tear.

A polymorphism is typically defined as two or more alternative sequences, or alleles, of a gene in a population. A polymorphic site is the location in the gene at which divergence in sequence occurs. Examples of the ways in which polymorphisms are manifested include restriction fragment length polymorphisms, variable number of tandem repeats, hypervariable regions, minisatellites, di- or multi-nucleotide repeats, insertion elements and nucleotide deletions, additions or substitutions. The first identified allele is usually referred to as the reference allele, or the wild type. Additional alleles are usually designated alternative or variant alleles. Herein, the sequences of the first aspect are designated the reference sequences, and are not part of the invention. Nucleic acid sequences of the present invention which differ from these sequences at one or more of the positions indicated above may be referred to as variants of these sequences.

A single nucleotide polymorphism is a variation in sequence between alleles at a site occupied by a single nucleotide residue. Single nucleotide polymorphisms (SNP's) arise from the substitution, deletion or insertion of a nucleotide residue at a polymorphic site. Typically, this results in the site of the variant sequence being occupied by any base other than the reference base. For example, where the reference sequences contains a “T” base at a polymorphic site, a variant may contain a “C”, “G” or “A” at that site.

The polymorphism of the invention occurs in a non-coding region and as such may not affect protein sequence, but may exert phenotypic effects by influencing replication, transcription and/or translation. A polymorphism may affect more than one phenotypic trait or may be related to a specific phenotype.

TABLE 1
INHBA02 SNP

| | | |
|---|---|---|
| 5′ | SNP | 3′ |

| | | | |
|---|---|---|---|
| INHBA02 | GTCATGAGAACTGGGCTTA | R | TTTCCAAAGTTCTTGATTTATGAAGCAGGCATTCAATTTCA |
| | TGCTTCAACTGTAGTCCTTC | | AGATGTAAAATAATAGTGTAAGTTGGTTGGGGTCGGGGG |
| | AAACAACCCTTCACCGTTCT | | AGACCATCATCATATGATTATACAGATTTTCCCAATTTTTT |
| | TGTCAGAGGAGCCCTTCCT | | TCCTCAATGAAATAGCTTATTTTAAAAATATAAAATTAGAA |
| | CCTGTGTTATTTACCAGACT | | TCCCTATAAAGAACACTACCAATTTTCTTTCCTGAAAGAA |
| | TGCCAACACCCCTCCCCCA | | ACAATAAAATACTTTGTAAATAGAAGCAACTTCATGAAGT |
| | CAAAAAAAGCCTGACCAGC | | AACAGATGTTTTATGTAAAGACATAAAACTGAACCTGAAT |
| | TTTACCCCTATTTCAAGGAG | | ATAAYGAGACGTGTGTGCGAGTAGTAAAAGTTGAAAGTT |
| | TGTTCACTTCTTTTCATTAAT | | GGACACACTCATTGAGACATATCTATTTGATTCCAATGTT |
| | CTGCTGTTATATTTGTAACA | | TTTCTAAAAGGTAGAGTAATCCTAGCCAGAGGTTTCACTG |
| | ATTTAGAAATTATAGGAGTT | | GCTCAGTGCATCACCCAGTAGTGTCTCAGAAGCCAGGAA |
| | GAAGTTCTGGTAAAAAAGA | | GGGCTTTCCATTAGATAATGAATTATGAAATGTCTCACAC |
| | ATGATGCATGAGGGTTTTT | | TGGAAAAACCAGTCATCCGCTGATGTCATGCTGATTCCA |
| | GTTGTTGTTGTTGTGTGTG | | ACCAATCCCAAACAAAGCCCCAGCCCTCCTCTGTTTCAG |
| | TGTGTGTGTGTGTGTGTGT | | TGGTACCAATGTGTGGTGTACAAATAAGTAGTACAGTATA |
| | GTTTGGTTAGTTAGAATGAG | | AAACTTCACAGTGCCAATACCATGAAGAGGAGCTCAGRC |
| | ATTCTAAAGACCTGGGAAG | | AGCTCTTACCACATGATACAAGAGCCGGCTGGTGGAAGA |
| | GATACTTATGAAGTTCATTT | | GTGGGGACCAGAAAGGTAATGCTTTTTAACTCTTACTTCT |
| | AAAGAGAATTACT | | GAGCTCTTTACACATTCAAAGATAGGAAAGCTAGGAGGA |
| | | | ATTTTACAACTAATTGGC |

wherein R represents the change of base of G to A.

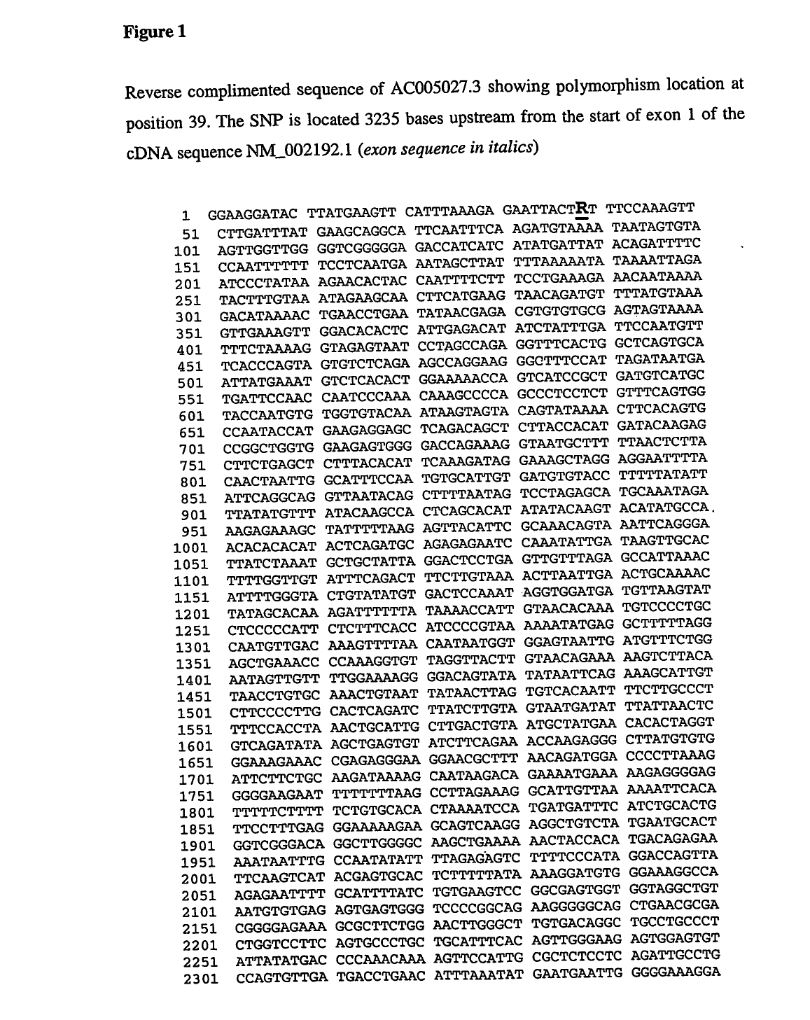

The method of the first aspect is carried out by methodology known to the person skilled in the art. Typically, the method involves contacting a sample of DNA, from a male or female individual, with an agent which identifies the presence or absence of a polymorphism described in Table land/or FIG. 1. Such an agent may be a hybridizing nucleotide, an antibody, a PCR agent or another agent. Further descriptions of agents are set out below, and methodology is described in the Examples section of this text.

The method may include determining whether one or more particular alleles are present, or which combination of alleles (i.e. a haplotype) is present. The method may also include determining whether subjects are homozygous or heterozygous for the particular allele or haplotype. In a preferred embodiment, the method comprises determining which allele of the polymorphism of the invention is/are present.

Any method, including those known to persons skilled in the art, may be used to determine which allele(s) of the INHBA gene are present. Preferably, the method comprises first removing a sample from a subject. More preferably, the method comprises obtaining an isolated sample of a polynucleotide to determine therein which allele(s) of the INHBA gene is/are present. Thus, the present invention relates to a non-invasive diagnostic method, the results of which provide an indication of susceptibility to bone damage but do not lead to a diagnosis upon which an immediate medical intervention is required.

Any biological sample comprising cells containing nucleic acid or protein is suitable for this purpose. Examples of suitable samples include whole blood, semen, saliva, tears, buccal, skin or hair. The sample and/or the nucleic acid may be immobilized on a support, such as a solid support.

In a preferred embodiment, the method is carried out using a polynucleotide. Any method for determining alleles in a polynucleotide may be used, including those known to persons skilled in the art. Preferably, the method may comprise the use of anti-sense polynucleotides, for example, as defined below. Such polynucleotides may be sequences which are able to distinguish between alleles of the INHBA gene, by preferential binding, or sequences which hybridise under stringent conditions to a region either side of an allele to enable amplification of one or more of the polymorphisms.

Methods of this embodiment include those known to persons skilled in the art, for example direct probing, allele specific hybridisation, PCR methodology including Allele Specific Amplification (ASA), Allele Specific Hybridisation, single base extension, Genetic Bit Analysis and RFLP, or direct sequencing. The appropriate restriction enzyme, will, of course, be dependent upon the polymorphism and restriction site, and will include those known to persons skilled in the art. Analysis of the digested fragments may be performed using any method in the art, for example gel analysis, or southern blots.

Determination of an allele of a polymorphism using direct probing typically involves the use of anti-sense sequences, for example as described in the third aspect of the invention. These may be prepared synthetically or by nick translation. The anti-sense probes may be suitably labelled using, for example, a radiolabel, enzyme label, fluoro-label, biotin-avidin label for subsequent visualization in, for example, a southern blot procedure. A labelled probe may be reacted with a sample DNA or RNA, and the areas of the DNA or RNA which carry complimentary sequences will hybridise to the probe, and become labelled themselves. The labelled areas may then be visualized, for example by autoradiography.

The above described methods may require amplification of the DNA sample from the subject, and this can be done by techniques known in the art, such as PCR. Other suitable amplification methods include ligase chain reaction (LCR), transcription amplification, self sustained sequence replication and nucleic acid based sequence amplification (NASBA). The latter two methods both involve isothermal reactions based on isothermal transcription which produce both single stranded RNA and double stranded DNA as the amplification products, in a ratio of 30 or 100 to 1, respectively.

Where it is desirable to identify the presence of multiple single nucleotide polymorphisms, or haplotypes, in a sample from a subject, it may be preferable to use arrays. The array may contain a number of probes, each designed to identify one or more of the above single nucleotide polymorphisms of the invention.

A second aspect provides fragments of the INHBA gene itself or sequences complimentary thereto. Such fragments are useful in the method according to the first aspect of the invention. They may be isolated or recombinant.

The polynucleotide of this invention is preferably DNA, or may be RNA or other options.

By “isolated” is meant a polynucleotide sequence which has been purified to a level sufficient to allow allelic discrimination. For example, an isolated sequence will be substantially free of any other DNA or protein product. Such isolated sequences may be obtained by PCR amplification, cloning techniques, or synthesis on a synthesiser. By recombinant is meant polynucleotides which have been recombined by the hand of a man.

Preferred fragments are from at least 10 up to 1000, more preferably 10 up to 100 nucleotides in length. More preferably, the fragments are from 10, 20, 30, 40, 50, 60, 70, 80 or 90 nucleotides in length. The fragments should comprise a sequence which includes the polymorphism.

According to the second aspect of the invention, the preferred fragments are complementary sequences of the INHBA gene which hybridise a portion of the nucleic acid sequence as given in FIG. 1. Such “anti-sense” sequences are useful as agents for the identification of male or female individuals having or being susceptible to bone damage.

The anti-sense sequences of the invention include those which hybridise to an allele (preferably the variant allele) of a polymorphism of the invention. These sequences are useful as probes. To be useful as a probe, the anti-sense sequence should bind preferentially one allele of one or more polymorphisms of the present invention and will, preferably, comprise the exact complement of one allele of one or more polymorphisms of the invention. Thus, for example, where the variant comprises an “A” residue at a polymorphic site, it is preferred that the anti-sense sequence will comprise a “T” residue. Such anti-sense sequences which are capable of specific hybridisation to detect a single base mis-match may be designed according to methods known in the art. Variation in the sequence of these anti-sense sequence is acceptable for the purposes of the present invention, provided that the ability of the anti-sense sequence to distinguish between alleles of a polymorphism is not compromised. Preferably, the anti-sense sequence will hybridize to the sequence of interest under stringent conditions which are defined below.

In relation to the present invention, “stringent conditions” refers to the washing conditions used in a hybridisation protocol. In general, the washing conditions should be a combination of temperature and salt concentration so that the denaturation temperature is approximately 5 to 20° C. below the calculated Tm of the nucleic acid under study. The Tm of a nucleic acid probe of 20 bases or less is calculated under standard conditions (1M NaCl) as [4(C×(G+C)+2(C×(A+T)], according to Wallace rules for short oligonucleotides. For longer DNA fragments, the nearest neighbour method, which combines solid thermodynamics and experimental data, may be used. The optimum salt and temperature conditions for hybridisation may be readily determined in preliminary experiments in which DNA samples immobilised on filters are hybridised to the probe of interest and then washed under conditions of different stringencies. While the conditions for PCR may differ from the standard conditions, the Tm may be used as a guide for the expected relative stability of the primers. For short primers of approximately 14 nucleotides, low annealing temperatures of around 44° C. to 50° C. are used. The temperature may be higher depending upon the base composition of the primer sequence used.

In a third aspect of the invention, the polynucleotides of the aforementioned aspects of the invention may be in the form of a vector, to enable the in vitro or in vivo expression of the polynucleotide sequence. The polynucleotides may be operably linked to one or more regulatory elements including a promoter; regions upstream or downstream of a promoter such as enhancers which regulate the activity of the promoter; an origin of replication; appropriate restriction sites to enable cloning of inserts adjacent to the polynucleotide sequence; markers, for example antibiotic resistance genes; ribosome binding sites: RNA splice sites and transcription termination regions; polymerisation sites; or any other element which may facilitate the cloning and/or expression of the polynucleotide sequence. Where two or more polynucleotides of the invention are introduced into the same vector, each may be controlled by its own regulatory sequences, or all sequences may be controlled by the same regulatory sequences. In the same manner, each sequence may comprise a 3′ polyadenylation site. The vectors may be introduced into microbial, yeast or animal DNA, either chromosomal or mitochondrial, or may exist independently as plasmids. Examples of suitable vendors will be known to persons skilled in the art and include pBluescript II, LambdaZap, and pCMV-Script (Stratagene Cloning Systems, La Jolla (USA)).

Appropriate regulatory elements, in particular, promoters will usually depend upon the host cell into which the expression vector is to be inserted. Where microbial host cells are used, promoters such as the lactose promoter system, trytophan (Trp) promoter system, (beta-lactamase promoter system or phage lambda promoter system are suitable. Where yeast cells are used, preferred promoters include alcohol dehydrogenase I or glycolytic promoters. In mammalian host cells, preferred promoters are those derived from immunoglobulin genes, SV40, Adenovirus, Bovine Papilloma virus etc. Suitable promoters for use in various host cells would be readily apparent to a person skilled in the art.

In a fourth aspect of the present invention, there is provided host cell comprising a polynucleotide according to any of the aforementioned aspects, for expression of the polynucleotide. The host cell may comprise an expression vector, or naked DNA encoding said polynucleotides. A wide variety of suitable host cells are available, both eukaryotic and prokaryotic. Examples include bacteria such as E. coli, yeast, filamentous fungi, insect cells, mammalian cells, preferably immortalised, such as mouse, CHO, HeLa, myeloma or Jurkat cell lines, human and monkey cell lines and derivatives thereof. Such host cells are useful in drug screening systems to identify agents for use in diagnosis or treatment of male or female individuals having, or being susceptible to bone damage.

The method by which said polynucleotides are introduced into a host cell will usually depend upon the nature of both the vector/DNA and the target cell, and will include those known to a person skilled in the art. Suitable known methods include fusion, conjugation, transfection, transduction, electroporation or injection, as described in Sambrook et al.

In a fifth aspect of the present invention, there are provided antibodies which react (optionally or specific for) with an antigen such as a polynucleotide of the second aspect. Antibodies can be made by the procedure set forth by standard procedures. Briefly, purified antigen can be injected into an animal in an amount and in intervals sufficient to elicit an immune response. Antibodies can either be purified directly, or spleen cells can be obtained from the animal. The cells are then fused with an immortal cell line and screened for antibody secretion. The antibodies can be used to screen DNA clone libraries for cells secreting the antigen. Those positive clones can then be sequenced. Preferably, the antigen being detected and/or used to generate a particular antibody will comprise a polynucleotide or fragment according to the second aspect.

The detection of binding of the antibody to the antigen in a sample may be assisted by methods known in the art, such as the use of a secondary antibody which binds to the first antibody, or a ligand. Immunoassays including immunofluorescence assays (IPA) and enzyme linked immunosorbet assays (ELISA) and immunoblotting may be used to detect the presence of the antigen. For example, where ELISA is used, the method may comprise binding the antibody to a substrate, contacting the bound antibody with the sample containing the antigen, contacting the above with a second antibody bound to a detectable moiety (typically an enzyme such as horse radish peroxidase or alkaline phosphatase), contacting the above with a substrate for the enzyme, and finally observing the colour change which is indicative of the presence of the antigen in the sample.

In a sixth aspect of the present invention, there is provided a transgenic non-human animal comprising a polynucleotide according to the second aspect of the invention. Transgenic non-human animals are useful for the analysis of the single nucleotide polymorphisms and their phenotypic effect. Expression of a polynucleotide of the invention in a transgenic non-human animal is usually achieved by operably linking the polynucleotide to a promoter and/or enhancer sequence, preferably to produce a vector as previously described, and introducing this into an embryonic stem cell of a host animal by microinjection techniques. The transgene construct should then undergo homologous recombination with the endogenous gene of the host. Those embryonic stem cells comprising the desired polynucleotide sequence may be selected, usually by monitoring expression of a marker gene, and used to generate a non-human transgenic animal. Preferred host animals include mice and other rodents.

In a preferred embodiment, the transgenic non-human animal may comprise an anti-sense nucleic acid sequence of the second aspect. The expression of an anti-sense sequence in a transgenic non-human animal may be useful in determining the effects of such sequences on high bone mineral density, or in neutralising deleterious effects of variant genes in an animal. Preferably, the host animal will be one, which is susceptible to bone damage. The condition may be naturally occurring or artificially induced.

The seventh aspect of the invention also provides the use of the transgenic non-human animal of the sixth aspect, in screening for an agent for use in the determination of a male or female individual having, or being susceptible to bone damage.

In some preferred embodiments, for example where the susceptibility to bone damage has been artificially induced, the transgenic non-human animal will be modulated to no longer express the corresponding endogenous INHBA gene. Such animals may be referred to as “knock out”. In some cases, it may be appropriate to modulate the expression of the endogenous genes, or express the polynucleotides of the present invention, in specific tissues. This approach removes viability problems if the expression of a gene is abolished or induced in all tissues.

In an eighth aspect of the present invention there is provided a method of screening for an agent for use in the determination of male or female individuals having, or being susceptible to, bone damage, said method comprising contacting a putative agent with a polynucleotide according to the second aspect of the present invention, and monitoring the reaction there between. Potential agents are those which react differently with a variant of the invention and a reference allele. The reference allele may be the wild-type allele (i.e. without a polymorphism). It is envisaged that the present method may be carried out by contacting a putative agent with a host cell or transgenic non-human animal comprising a polynucleotide or protein according to the invention. Putative agents will include those known to persons skilled in the art, and include chemical or biological compounds, such as anti-sense polynucleotide sequences complementary to the coding sequences of the first aspect, or polyclonal or monoclonal antibodies which bind to a product such as a protein or protein fragment of the second aspect. They may also be useful in determining susceptibility to bone damage, or in the diagnosis, prognosis or treatment of related conditions.

A ninth aspect of the invention provides primer sequences suitable for PCR reactions, for use in determining the presence or absence of a polymorphism as described herein. Suitable sequences should comprise at least 18 nucleotide bases and may be one or more sequences, selected from:

any sequence which hybridizes to the INHBA gene. The primer may be 100% complimentary to the INHBA sequence or may be 80% or more complimentary. Preferably the sequence is from 18 to 25 bases in length.

In a further aspect, the present invention provides a kit for use in diagnosis of a male or female individual having or being susceptible to bone damage, the kit comprising an agent for determining the presence or absence of the polymorphism described herein. The agent of the kit may comprise polynucleotides, mostly preferably anti-sense sequences such as those of the second aspect, for use as probes or primers; sequences of the eighth aspect of the invention; antibodies which bind to alleles of the INHBA polypeptides, such as those of the fifth aspect; or restriction enzymes for use in detecting the presence of a INHBA polynucleotide. Preferably, the kit will also comprise means for detection of a reaction, such as nucleotide label detection means, labelled secondary antibodies or size detection means. In yet a further preferred embodiment, the agent may be fixed to a substrate, for example an array, as described in WO95/11995. The kit further comprises means for indicating correlation between the genotype of a subject and susceptibility to high bone mineral density. Such means may be in the form of a chart or visual aid, which indicates that presence of one or more alleles of the INHBA gene, is associated with susceptibility to bone damage.

An eleventh aspect of the present invention provides a method for diagnosing and then acting on any diagnosis of the polymorphism described hereinto prevent and/or treat bone damage. The prevention and/or treatment may be by any means, including replacing the allele with the polymorphism. The replacement may be by addition of an allele, or part thereof, with a polynucleotide of the INHBA gene which is not associated with bone damage.

The preferred embodiments of each aspect apply to the other aspects of the invention, mutatis mutandis.

The present invention will now be described by way of a non-limiting example, with reference to the figures in which:

FIG. 1 Nucleotide sequence showing position of the associated single nucleotide polymorphism

### EXAMPLE 1

Biallelic Polymorphism Genotyping by Pyrosequencing™

A pair of oligonucleotides for amplification by PCR was designed on either side of the biallelic polymorphism to produce a product size between 50 bp and 350 bp. A sequencing oligonucleotide was designed to end within 30 bp either 5′ or 3′ to the polymorphic site. All amplification oligonucleotides used to generate the complementary strand to the sequencing primer were labelled with a 5′-Biotin (see Table 2).

TABLE 2

PSO assay oligonucleotides and
PCR annealing temperatures

| | Unlabelled | Biotin | Sequencing | |
|---|---|---|---|---|
| Polymorphism | PCR oligo- | PCR oligo- | oligo- | TA |
| ID | nucleotide | nucleotide | nucleotide | ° C. |
| INHBA G-692A | GACCCCAACCAA | GAGATTCTAAAGAC | CATAAATCAAGAA | 54 |
| | CTTACAC | CTGGGAAG | CTTTGGA | |

The sample genotyped was amplified by PCR using the PCR amplification oligonucleotides. Each reaction used: 20 ng DNA (dried down), 0.6 units of AmpliTaq Gold™ DNA polymerase, 1×PCT Buffer II, 2.5 mM MgCl₂, 1 mM dNTP, and 10 pmol of each PCR oligonucleotide in a final volume of 10 μl. The PCR cycling conditions used were: 95° C. for 12 min, 45 cycles of: 94° C. for 15 sec, T_{A} for 15 sec, 72° C. for 30 sec, and 72° C. for 5 min.

After amplification the DNA strand of each PCR template complementary to the sequencing primer was isolated, ready for pyrosequencing (PSQ). To do this, 1) 50 μl of Dynabead solution (2 mg/ml Dynabeads®, 5 mM Tris-HCl, 1M NaCl, 0.5 mM EDTA, 0.05% Tween 20) was added to the PCR product and shaken at 65° C. for 15 min, 2) the template was transferred using magnets to 50 μl of 0.5M MaOH for 1 min, 3) the template was transferred using magnets to 100 μl of 1× Annealing buffer (20 mM Tris-Acetate, 5 mM MgAc₂) for 1 min, and 4) the template was transferred using magnets to 45 μl of 1× Annealing buffer containing 15 pmol of sequencing oligonucleotide (Table 4).

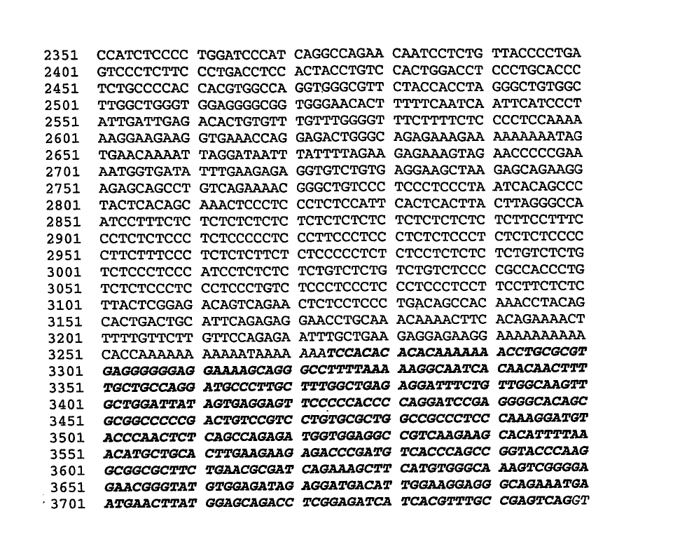

After template isolation, the sequencing oligonucleotide was annealed to the template by denaturing at 80° C. for 2 min and then cooling to room temperature for 10 min. Each marker/sample combination was then sequenced/genotyped by pyrosequencing™ on a PSQ96™ (Pyrosequencing AB) (FIG. 3). Genotype results were stored in the PSQ oracle® database ready for statistical analysis.

### EXAMPLE 2

INHBA Gene Polymorphism in Determining Genetic Susceptibility to Osteoporotic Fracture.

A set of pedigrees, initially collected for genome wide linkage analysis for bone mineral density, was used to test for transmission distortion at the INHBA02 SNP using the program transmit (A generalization of the transmission/disequilibrium test for uncertain-haplotype transmission. Clayton D. Am J Hum Genet 1999 October; 65(4): 1170-7). Affection status was defined as any self reported fracture at age greater than fifty in females. Results are given in Table 3.

TABLE 3
Transmission distortion of INHBA02 to females, with self reported
fracture at age >50.

| | |
|---|---|
| Rare allele frequency | 0.319 |
| Observed transmissions, rare allele | 104 |
| Expected transmissions, rare allele | 92.33 |
| Observed transmissions, common allele | 166 |
| Expected transmissions, common allele | 177.67 |
| Probability | 0.003 |
| Number of families included in the analysis | 815 |
| Number of affected offspring | 135 |

Number of families with transmissions to affected offspring 123

These results demonstrate highly significant transmission distortion, indicating that the less common allele (the “A” allele) is associated with an increased risk of fracture in females at an age greater than 50.

### EXAMPLE 3

SNP Association to Bone Mineral Density

The genotypes of 2,812 individuals from a set of pedigrees initially collected for genome wide linkage analysis for bone mineral density was used to test for association to bone mineral density (BMD) at the INHBA02 SNP using logistic regression. Adjusted and unadjusted BMD measurements are shown below for lumbar spine.

Allele 1 (A) of INHBA02 is associated with a significant decrease in unadjusted lumbar spine BMD (p=0.034, magnitude of effect=−2.1%) and adjusted lumbar spine BMD (p=0.035, magnitude of effect=−0.121) in males.

| | Unadjusted BMD | Adjusted BMD |
|---|---|---|
| Marker | Lumbar spine | Lumbar-spine |
| mean | 1.083 | −0.027 |
| heritability | 0.736 | 0.729 |
| INHA02 | 0.034 | 0.035 |

1. A method of determining susceptibility to bone damage, comprising determining the presence or absence of the polymorphism at position 39 of at least one allele of the INHBA gene in a male or female individual.

2. A method as claimed in claim 1 wherein the polymorphism is the presence of nucleotide base A.

3. An isolated or recombinant polynucleotide comprising from at least 10 to 1000 consecutive nucleotide bases of the INHBA sequence, which sequence comprises a nucleotide at position 39.

4. An isolated or recombinant polynucleotide as claimed in claim 3 comprising from at least 10 to 100 consecutive nucleotide bases of the INHBA sequence, which sequence comprises a nucleotide at position 39.

5. A polynucleotide, as claimed in claim 3 wherein the nucleotide at position 39 is not the nucleotide G.

6. A polynucleotide, as claimed in claim 5, wherein the nucleotide at position 39 is the nucleotide A.

7. A vector comprising a polynucleotide according to claim 3.

8. A host cell comprising a polynucleotide or vector according to claim 3.

9. An antibody or antibody fragment which preferentially binds to a sequence, as claimed in claim 3.

10. A transgenic non-human animal comprising a polynucleotide sequence, vector or host cell according to claim 3.

11. A method of determining susceptibility to bone damage, comprising determining the presence or absence of the polymorphism at position 39 of at least one allele of the INHBA gene in a male or female individual, which comprises the use of a polynucleotide, antibody or antibody fragment, as claimed in claim 3.

12. Use of a transgenic non-human animal according to claim 10 in screening for an agent for use in the determination of a male or female individual having, or being susceptible to bone damage, including bone fracture.

13. A method of screening for an agent for use in the determination of a male or female individual having, or being susceptible to bone damage, said method comprising contacting a putative agent with a polynucleotide, as claimed claim 3 and monitoring the reaction there between.

14. A polynucleotide which comprises a nucleic acid sequence of at least 18 bases which can be used to amplify, by PCR, a portion of the INHBA gene which comprises position 39.

15. A kit for use in diagnosis of a male or female individual having, or being susceptible to bone damage, said kit comprising an agent for determining the presence or absence of a polymorphism of at least one allele at position 39.

16. A kit for use in diagnosis of a male or female individual having, or being susceptible to bone damage, said kit comprising an agent for determining the presence or absence of a polymorphism of at least one allele at position 39, wherein the agent comprises a polynucleotide according to claim 3.

17. A kit for use in diagnosis of a male or female individual having, or being susceptible to bone damage, said kit comprising an agent for determining the presence or absence of a polymorphism of at least one allele at position 39, wherein the agent is identified according to a method of claim 13.

18. A method for diagnosing and preventing and/or treating bone damage, the method comprising 1) determining the presence of a polymorphism at position 39 in at least one allele of the INHBA gene in a male or female individual; and 2) administering to the individual an agent which prevents and/or treats bone damage.

19. A kit for use in diagnosis of a male or female individual having, or being susceptible to bone damage, said kit comprising an agent for determining the presence or absence of a polymorphism of at least one allele at position 39, wherein the agent comprises an antibody according to claim 9.

20. A kit for use in diagnosis of a male or female individual having, or being susceptible to bone damage, said kit comprising an agent for determining the presence or absence of a polymorphism of at least one allele at position 39, wherein the agent comprises restriction enzymes for digestion of a polynucleotide according to claim 3.

21. A kit for use in diagnosis of a male or female individual having, or being susceptible to bone damage, said kit comprising an agent for determining the presence or absence of a polymorphism of at least one allele at position 39, wherein the agent comprises restriction enzymes for digestion of a polynucleotide according to claim 14.
